(19) United States
(12) Patent Application Publication
(10) Pub. No.: US 2023/0097858 A1
(43) Pub. Date: Mar. 30, 2023

(54) SYSTEMS AND METHODS FOR MOLECULAR MEASUREMENTS

(71) Applicant: CALIFORNIA INSTITUTE OF TECHNOLOGY, Pasadena, CA (US)

(72) Inventor: Axel SCHERER, Barnard, VT (US)

(21) Appl. No.: 17/937,178

(22) Filed: Sep. 30, 2022

Related U.S. Application Data
(60) Provisional application No. 63/277,033, filed on Nov. 8, 2021.
(60) Provisional application No. 63/250,340, filed on Sep. 30, 2021.

(51) Int. Cl. G01N 27/04; G01N 27/22; C12Q 1/6869

(52) U.S. Cl. CPC G01N 27/04; G01N 27/22; C12Q 1/6869

(57) ABSTRACT

Systems and methods for identifying the components of a long-chain molecule by making electrical measurements from fabricated nanoscale electrodes as the molecule moves down a narrow microfluidic channel. The channel can be along the surface of a chip, through a chip, or both.

### CROSS-REFERENCE TO RELATED APPLICATIONS

The present application is related to U.S. Provisional Patent Application No. 63/250,340 filed on Sep. 30, 2021 and U.S. Provisional Patent Application No. 63/277,033 filed on Nov. 8, 2021, the disclosures of which are incorporated herein by reference in their entirety.

### BACKGROUND

The definition of small contacts and electrodes through lithographic fabrication typically relies on using a high-resolution lithographic instrument and performing additive or subtractive processing to pattern an electrically conducting material onto an insulating surface. This process scales well down to 5 nm but is very difficult to use to define conducting structures with smaller dimensions below 2 nm. Electrode structures with molecular dimensions are of great interest in many biochemical and electronic applications.

One of the most formidable challenges in biochemistry is the sequencing of DNA, RNA and protein molecules. This task has become the goal of a sizeable industry developing expensive instrumentation, measuring nucleotide sequences mainly through optical and electrochemical signals. Many approaches have been pursued, and expensive and large sequencers are used for many applications ranging from oncology to the identification of virus strains. Approaches to reduce the size of sequencing systems have in the past focused on the interrogation of nucleic acid molecules through membranes with bio-functionalized pores (Oxford Nanopore™), or by monitoring many parallel electrochemical reactions (Ion Torrent™), and miniaturized geometries that are fabricated with excellent precision by using lithographic approaches have become available. These systems still require expensive and large electronic amplifiers, or high-sensitivity optical cameras to be useful.

### SUMMARY

Some embodiments herein describe geometries that leverage microelectronic fabrication techniques and are modified to interrogate molecules as they flow through nano-fluidic circuits. One distinction between the structures described herein and previous efforts is that direct electrical conductivity and capacitance measurements can be conducted on the molecules of interest, and signals obtained from this interrogation can be amplified and interpreted by the surrounding CMOS electronics without the need of additional amplifier or detection electronics. This approach enables the overall size of sequencing systems to be radically reduced and results in a new class of integrated nanofluidic CMOS electronics devices.

In a first aspect of the disclosure, a sensor for sequencing a long-chain molecule is disclosed, the sensor comprising: one or more electrodes, each electrode essentially consisting of a conductive layer and an insulator layer, the conductive layer and the insulator layer being less than 4 nm thick each; a microfluidic channel configured to present the long-chain molecule to the one or more electrodes such that the long-chain molecule can be sequenced by measurements taken by the one or more electrodes.

In a second aspect of the disclosure, a method of sequencing a long-chain molecule is disclosed, the method comprising: passing the long-chain molecule through a microfluidic channel that is adjacent to a conductive layer of less than 4 nm thickness; taking an electrical measurement from the conductive layer as the long-chain molecule passes the conductive layer; and determining a component of the long-chain molecule from the electrical measurement.

In a third aspect of the disclosure, a method of fabricating sensor for sequencing a long-chain molecule is disclosed, the method comprising: depositing one or more electrodes, each electrode essentially consisting of a conductive layer and an insulator layer, the conductive layer and the insulator layer being less than 2 nm thick each; fabricating a microfluidic channel configured to present the long-chain molecule to the one or more electrodes such that the long-chain molecule can be sequenced by measurements taken by the one or more electrodes.

In a fourth aspect of the disclosure, a sensor for sequencing a long-chain molecule, the sensor comprising: a fin field effect transistor, the source and drain being fins less than 1 nm thick and the gate having a microfluidic channel etched through it perpendicular to the fins.

### BRIEF DESCRIPTION OF DRAWINGS

FIG. 1 shows an example of metal-insulator multi-layer deposition to produce nanoscale electrodes.

FIG. 2 shows example views (side and perspective) of the nanoscale electrode structure produced in FIG. 1.

FIG. 3 shows an example process of creating nanoscale electrodes with contacts.

FIG. 4 shows an example of nanoscale electrodes integrated with a microfluidic channel tangent to the structure surface.

FIG. 5 shows an example of nanoscale electrodes with a built-in microfluidic channel through the structure, perpendicular to the surface.

FIG. 6 shows an example of using nanoscale electrodes with a microfluidic channel to make three types of measurements on a molecule.

FIG. 7 shows an example four-segment nanoscale electrode block with a built-in microfluidic channel at the juncture of the segments.

FIG. 8 shows an image of a 5 nm microfluidic channel with corresponding perspective and side (cross-sectional) views.

FIG. 9 shows an example of nanoscale electrodes with microfluidic channels connected by offset holes.

FIGS. 10A and 10B show a FINFET transistor with integrated microfluidic channel. FIG. 10A shows the FINFET without the channel, FIG. 10B shows the FINFET with the channel.

FIGS. 11A-11F show an example fabrication for a capacitance sensor using nanoscale electrodes with microfluidic channels.

FIGS. 12A-12E shows an example of angle deposition used for creating nanoscale electrodes for microfluidic measurements.

### DETAILED DESCRIPTION

Presented herein, nanoscale thickness wafer/chip deposition techniques in geometries that connect to or provide microfluidic paths can produce sensors that can identify portions of long-chain molecules through electronic detection.

The three-dimensional nature of geometries that are routinely fabricated on a wafer-scale is uniquely suited to enable the measurement of molecules (e.g. DNA) without the need of indirect fluorescent or electrochemical measurements that suffer from errors and require very sensitive optics or electronics.

As used herein, “nanoscale” refers to sizes on the scale of around 1 nm, and for the purposes of this disclosure and claims can range from greater than 0 nm to less than 4 nm.

As used herein, “interrogate” means to take electrical measurements of an object in order to determine the composition of the object. Electrical measurements include capacitance, resistance (conductivity), response to signals, and similar electrical properties of matter. The composition of the object can be determined by comparing the electrical measurement against known values for various objects (e.g. DNA/RNA bases or amino acids having different sizes/chemical compositions). As a chain of objects (e.g. nucleotides in DNA) move past the sensor's electrodes, the interrogation can build up an identification of a sequence of those objects for the chain. A device used for interrogation can be referred to as a “sensor”.

As used herein, “long-chain” refers to a molecule with at least 10 repeating unit parts (base pairs, amino acids, carbon atoms, etc.).

Two general configurations for these systems can be categorized as “lateral” and “vertical”. A lateral system provides electrode contact to a microfluidic path going across a surface or parallel to the width of the device (e.g. wafer). A vertical system provides electrode contact to a microfluidic path going through the device. Some devices are combinations of these systems, with the microfluidic path going both laterally and vertically.

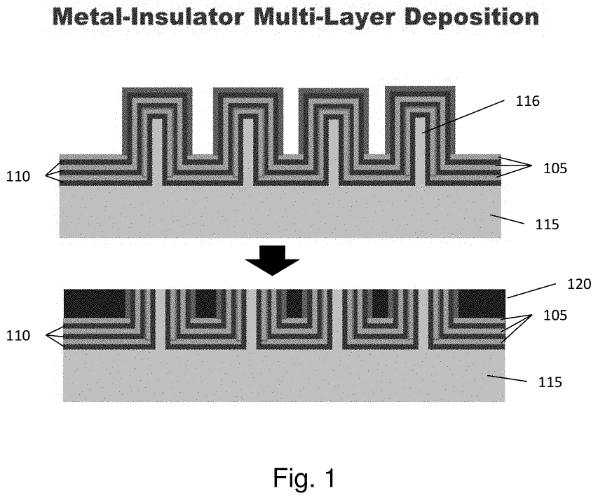

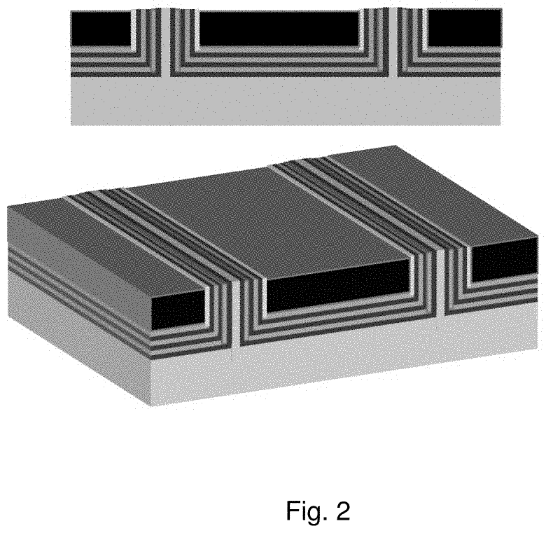

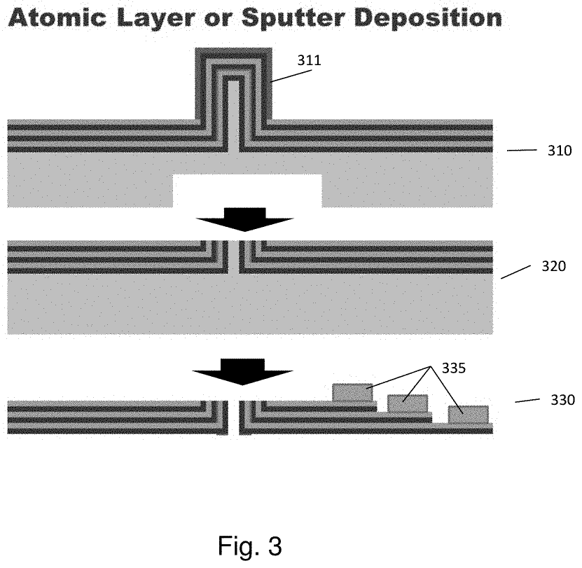

FIG. 1 shows an example of deposition of metal and insulator material in layers to provide nanoscale thick layers for electrodes. In some embodiments, the layers are less than 2 nm thick. In some embodiments, the layers are less than 4 nm thick. Alternating layers of insulator 105 and conductive material 110 are deposited on a substrate 115 by a conformal deposition technique (e.g. atomic layer epitaxy, chemical vapor deposition, sputter deposition) that provides a controlled and uniform thickness to the layers at the nanoscale size. If the system involves lateral measurements, the layers can be deposited on nanopillars 116, such that when the wafer is back-filled 120 with, for example, an insulating material and milled/polished flat, the nanoscale layers contact the surface of the wafer to allow contact to a microfluidic path along that surface and short distances between the electrodes (defined by the thickness of the nanopillar). FIG. 2 shows the resulting electrode system in cross-section and perspective views. FIG. 3 shows a similar system where there is deposition 310 over a nanopillar 311, then it is milled down 320, and finally electrical contacts 335 are attached 330. As shown, the conducting electrode layers in these structures can be separately connected using a fan-out approach to enable separate contacts to the conductors, as long as the insulator layers are not compromised.

The conductive layers can be any conductive material (typically metal) appropriate for the application. Examples include platinum, gold, conductive forms of carbon (e.g. graphene), or various composites/alloys. The insulating materials can be an oxide, such as TaO₂, HfO₂, or Al₂O₃, or a fluoride, or a polymer. In some embodiments, the layering is insulator over conductive layer repeating, in some embodiments it is conductive layer over insulator repeating. The number of layer pairs can vary depending on the application, from one pair on up.

Some embodiments show the geometries of different measurement approaches, which can be implemented, for example, on a silicon CMOS (complementary metal-oxide semiconductor) geometry or the like. The flow channels can be fabricated either in the semiconductor or the oxide layer, or in the via-holes that are typically defined to provide contacts for transistors. To form appropriate channels, one can selectively remove materials with atomic precision. Many chemical and gas-based techniques have been developed to perform this task, and these can be implemented on a wafer-scale in commercial semiconductor fabrication lines. Small changes to the CMOS process are necessary to accommodate the needs for nanofluidic channels for particular applications.

For example, the choice of metals in the metallization layers, which is usually either copper or aluminum, is not ideal for biochemical measurements, and therefore these metals would have to be replaced with metals that perform the desired interaction with DNA. For example, it is well known that gold can be used as a metal for thiol-bonding to cysteine, and this metal can be incorporated into the channels to perform that specific task. Platinum is known for its electrochemical stability, and it is useful to incorporate it when electrochemical and conductivity measurements are performed, as it is galvanically predictable. Carbon in its graphite, graphene or nano-tube forms is another material that can be used to develop favorable chemical properties to measure or manipulate nucleic acid molecules, as it is known to be very bio-compatible. These conductors can be used to replace copper and aluminum, either universally or just locally around the nanofluidic channel, along with the intermetallic silicide (TaSi₂, MoSi₂, WSi₂, etc.) contact and diffusion control layers that are commonly used in silicon CMOS chips.

Milling down the nanopillars can be performed by techniques known in the art, such as mechanical polishing, angle ion milling, or etching.

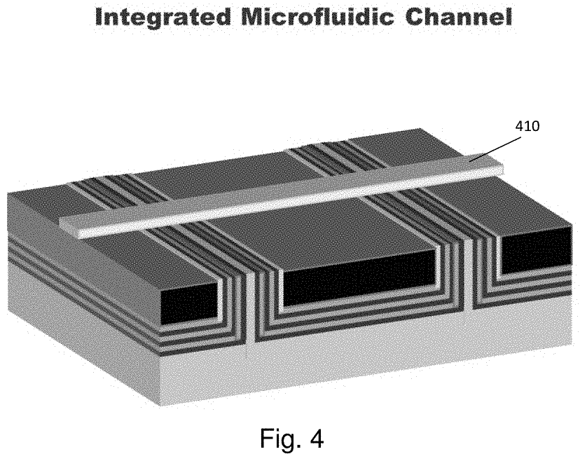

FIG. 4 shows an example of a lateral sensor system, with a microfluidic path 410 situated over the electrodes. As molecules traverse down the path 410, the system can measure resistance and/or capacitance values across the electrodes for different segments along the molecule, which would mainly be confined to be along the direction of the path. Since the electrodes are nanoscale in scale, these measurements would determine the constituent parts of the molecule. In the case of DNA, RNA, or proteins, this would allow the sequencing of the bases/nucleotides/amino acids of those molecules in real-time. Multiple measurements can be taken to increase the reliability of the sequencing through statistical methods.

In some embodiments, the microfluidic path has a lateral dimension in the nanometer scale (<1 micrometer). In some embodiments, the microfluidic path has a lateral dimension in the micrometer scale (>=1 micrometer). The vertical dimension of the microfluidic path is preferably small (to take advantage of the narrow electrode spacing). In some embodiments, the vertical dimension is less than 20 nm.

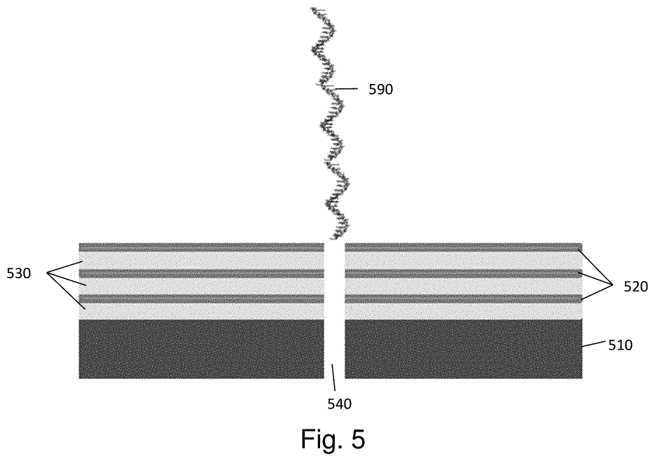

FIG. 5 shows an example of a vertical system. In this example, the metal 530 and insulator 520 layers are deposited without a nanopillar, but a channel 540 is etched into the sensor to provide a microfluidic path through the layers. The channel 540 is sized to allow a long-chain molecule of interest 590 through, but only in a vertical orientation. As the molecule 590 passes by the electrodes (520, 530), they can measure the resistance and/or capacitance of the constituent parts of the molecule to allow sequencing of that molecule.

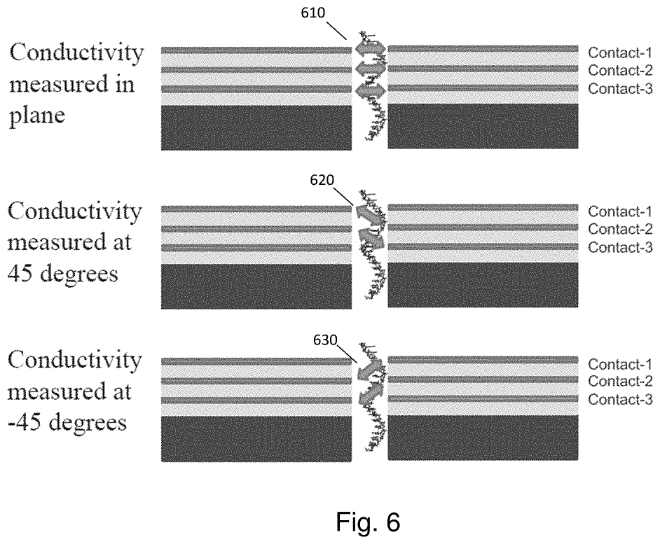

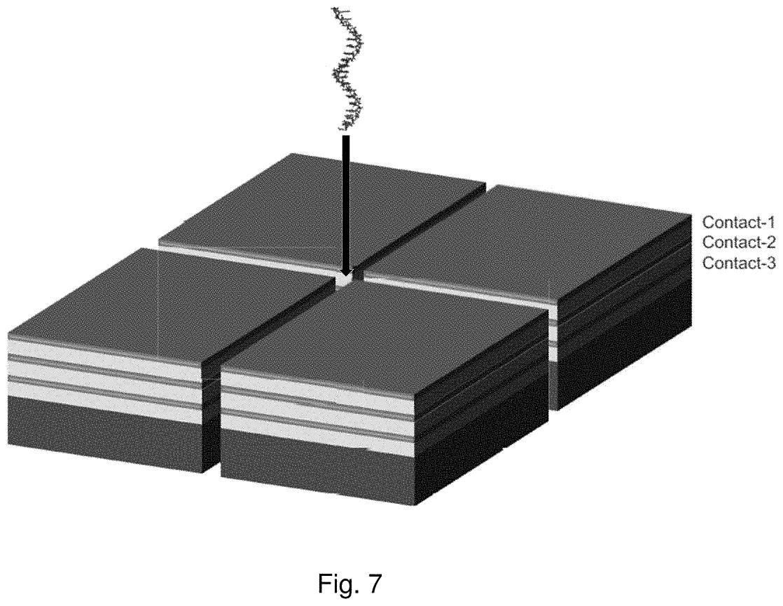

In some embodiments, a method of tomography (cross-sectional analysis) of a molecule can be made by making multiple measurements at different geometries. Instead of performing simple measurements perpendicular to the flowing molecule, it is also possible to perform measurements along different directions, determined by the arrangement of the electrodes surrounding the channel, providing tomographic 3D information of the segment of the molecule that passes through the interrogation section. As the dimensions of the lithographic electrode contacts would be much smaller than the molecule, it is also possible to measure the same molecule several times in slightly different geometries, correlating the measurements in different segments of the interrogation channel (the portion of the microfluidic channel passing by the electrodes). As shown in the example of FIG. 6, measurements can be made in-plane 610, at a 45 degree angle 620, and at −45 degrees 630 by utilizing different sets of opposing contacts in combination. Other combinations and angles can be realized as well, based on the configuration of the layers. The combination of these measurements can be used to create a tomographic 3D rendering of the molecule, or of a part of the molecule. FIG. 7 shows an example basic geometry for use in tomography. The tomographic technique is conceptually the same as used in medical imaging (of organs, bones, joints, etc.), but scaled down to the nanoscale level (molecules).

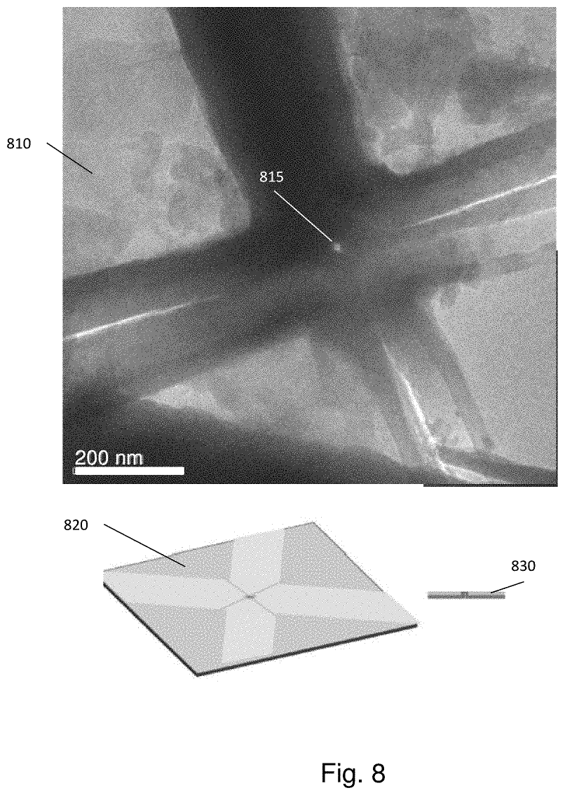

FIG. 8 shows an example image of a vertical system 810 with a nanopore 815 (here 5 nm across) to create the microfluidic channel. A perspective view 820 and cross-sectional view 830 are also shown.

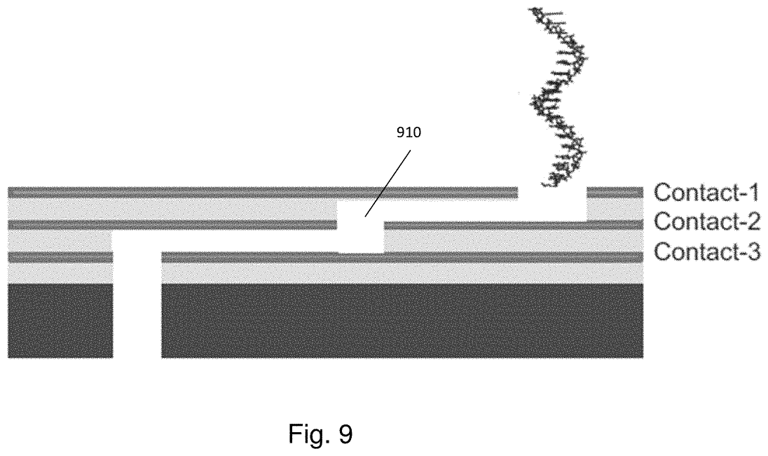

FIG. 9 shows an example of a system with a series of channels connected by offset holes between layers, for both capacitance and conductivity (resistance) measurements. The channel 910 can be realized with known etching techniques.

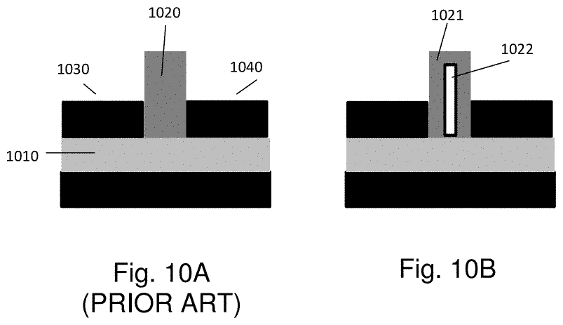

FIGS. 10A and 10B illustrate an example of a system utilizing a FinFET (fin field effect transistor). FIG. 10A shows an example configuration of a FinFET, with a source 1030, gate 1020, and drain 1040 on a substrate 1010. The source 1030 and drain 1040 being in a “fin” configuration at less than 1 nm thick. FIG. 10B shows a FinFET modified for the system, with a microfluidic channel 1022 etched/fabricated through the gate 1020, to allow electrical interrogation of the molecules that pass through. An example would by XeF₂ etching. Other FET configurations with elements on the nanometer scale can be realized with the addition of a microfluidic channel.

The converted FinFET can then be used as a microfluidic channel surrounded by nanoscale electrodes (the source and drain). As the FinFET structure has already been proven to be manufacturable, structures like it can be used for nanofluidic applications by using surface micromachining approaches. Converting a FinFET into an ion-sensitive FET is a relatively simple way of leveraging a known three-dimensional electronic device into a fluidic structure.

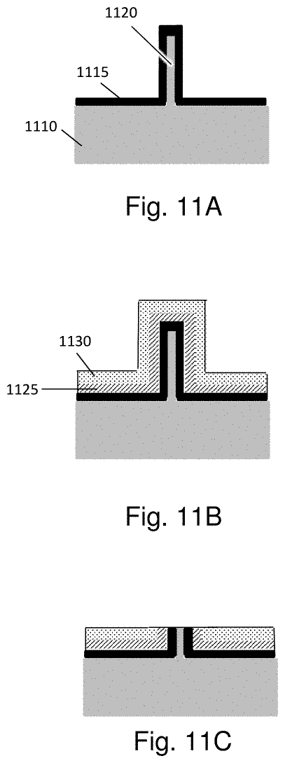

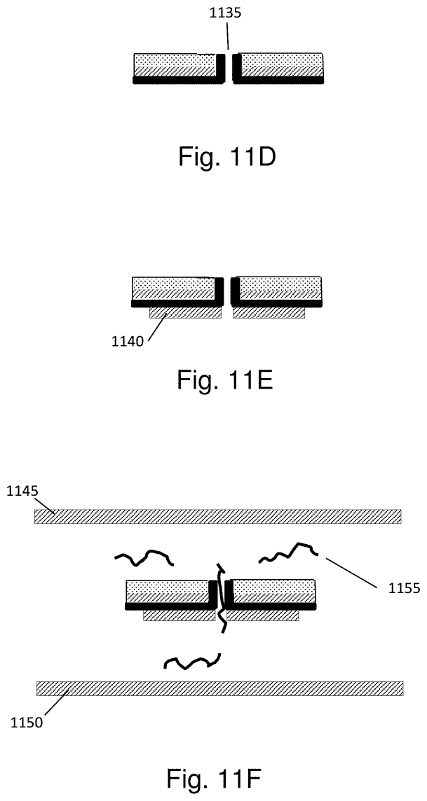

FIGS. 11A-11F show an example of a fabrication of a capacitance sensor. FIG. 11A shows a substrate 1110 (e.g. Si) with a nanopillar 1120 all covered in an oxide 1115 (e.g. SiO₂). FIG. 11B shows the deposition (nanoscale) of metal 1125 and insulator 1130 layers. FIG. 11C shows the nanopillar milled down. FIG. 11D shows the substrate removed to provide a channel 1135 vertically through the sensor. FIG. 11E shows the addition of back metal contacts 1140. FIG. 11F shows the sensor between negative 1145 and positive 1150 electrical contacts, where molecules 1155 (e.g. DNA) can flow through the channel for capacitive interrogation.

The measurement of capacitance is through a capacitance bridge circuit, in which measurements of the fluid channel are performed at different frequencies in parallel with a similar capacitor without a fluid channel (a reference signal). The two capacitances are in a circuit that subtracts one from the other, and the difference of the two capacitances can then be amplified with an analog background subtraction system. This difference can then be compared to known values of different molecule elements (e.g. bases or amino acids) to identify the structure of the molecule.

### EXAMPLE

### Angle Deposition

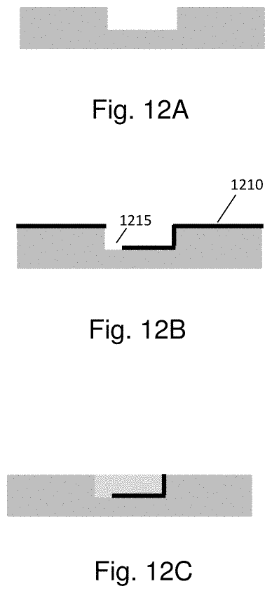

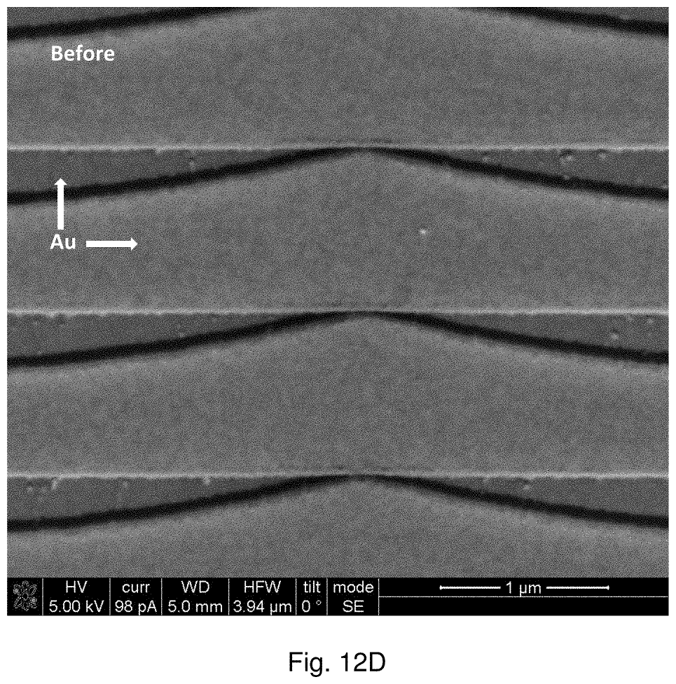

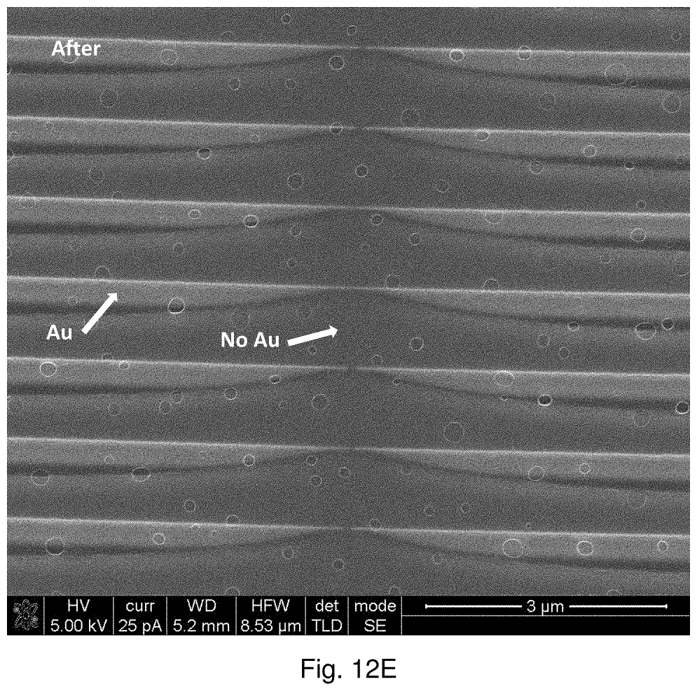

In some embodiments, the nanoscale electrodes can be fabricated by angle deposition and etching techniques. As an example, a device can be initially patterned as shown in FIG. 12A using electron beam lithography (e.g. using ZEP520A™ resist), using etching techniques (e.g. Cr etch, ICP-RIE etch) and resist removal (e.g. acetone and oxygen plasma). As shown in FIG. 12B, a conductive layer 1210 (e.g. Ti—Au) can be angle deposited, which leaves a gap 1215 for a possible placement of a vertical or lateral microfluidic channel (alternatively, or in combination, a surface channel can be used in the lateral configuration). The top can be spin-coated with poly(methyl methacrylate) and then ion milled down as needed, as shown for example in FIG. 12C.

FIGS. 12D and 12E show images of the pattern formed in this example before (FIG. 12D) and after (FIG. 12E) ion milling, showing the final result of exposed electrodes (Au) on the nanometer scale. In this example, the dose is 160 μC/cm² with a pattern having a narrow dimension of 30 nm. The milling parameters were 20 standard cubic cm per minute (sccm) Ar flow at 9.6E-5 Torr, cathode at 14.7 volts and 17.7 amps, beam at 500 volts and 120 mA, milled at a 45° angle for 22 seconds. The height of the angle deposited conductive layer can be modulated by changing the width of the patterned channel (see FIG. 12A) that is used for the angle deposition process.

A number of embodiments of the disclosure have been described. Nevertheless, it will be understood that various modifications may be made without departing from the spirit and scope of the present disclosure. Accordingly, other embodiments are within the scope of the following claims.

The examples set forth above are provided to those of ordinary skill in the art as a complete disclosure and description of how to make and use the embodiments of the disclosure, and are not intended to limit the scope of what the inventor/inventors regard as their disclosure.

Modifications of the above-described modes for carrying out the methods and systems herein disclosed that are obvious to persons of skill in the art are intended to be within the scope of the following claims. All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

It is to be understood that the disclosure is not limited to particular methods or systems, which can, of course, vary. It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

The following are claimed:

1. A sensor for sequencing a long-chain molecule, the sensor comprising:
one or more electrodes, each electrode essentially consisting of a conductive layer and an insulator layer, the conductive layer and the insulator layer being less than 4 nm thick each;
a microfluidic channel configured to present the long-chain molecule to the one or more electrodes such that the long-chain molecule can be sequenced by measurements taken by the one or more electrodes.

2. The sensor of claim 1, wherein the measurements comprise electrical resistance, capacitance, or both.

3. The sensor of claim 1, wherein the conductive layer is comprised of gold.

4. The sensor of claim 1, wherein the conductive layer is comprised of platinum.

5. The sensor of claim 1, wherein the conductive layer is comprised of graphite, graphene, or carbon nano-tubes.

6. The sensor of claim 1, wherein the sensor has a surface and the microfluidic channel is situated laterally on the surface and passes over the electrodes.

7. The sensor of claim 1, wherein the microfluidic channel goes through the electrodes.

8. A method of sequencing a long-chain molecule, the method comprising:
passing the long-chain molecule through a microfluidic channel that is adjacent to a conductive layer of less than 4 nm thickness;
taking an electrical measurement from the conductive layer as the long-chain molecule passes the conductive layer; and
determining a component of the long-chain molecule from the electrical measurement.

9. The method of claim 8, wherein the long-chain molecule is DNA or RNA and the component is a nucleotide.

10. The method of claim 8, further comprising taking a second electrical measurement at a different geometry than the electrical measurement and forming a tomographic 3D rendering of the long-chain molecule.

11. The method of claim 8, wherein the electrical measurement is at least one of resistance or capacitance.

12. The method of claim 8, further comprising taking a second electrical measurement from the conductive layer as a second long-chain molecule passes the conductive layer, the second long-chain molecule being a copy of the long-chain molecule, and using statistical methods for the determining the component based on both the electrical measurement and the second electrical measurement.

13. The method of claim 8, wherein the electrical measurement is performed while the conductive layer and the microfluidic channel are placed between a negative electrical contact and a positive electrical contact.

14. A method of fabricating a sensor for sequencing a long-chain molecule, the method comprising:
depositing one or more electrodes, each electrode essentially consisting of a conductive layer and an insulator layer, the conductive layer and the insulator layer being less than 2 nm thick each;
fabricating a microfluidic channel configured to present the long-chain molecule to the one or more electrodes such that the long-chain molecule can be sequenced by measurements taken by the one or more electrodes.

15. The method of claim 14, wherein the fabricating the microfluidic channel comprises etching a pore through the one or more electrodes.

16. The method of claim 14, further comprising forming a nanopillar from a substrate that the one or more electrodes will be deposited on and milling down the nanopillar after the one or more electrodes are deposited.

17. The method of claim 16, further comprising removing the substrate and the nanopillar to form the microfluidic channel.

18. The method of claim 16, further comprising back-filling over the one or more electrodes with an insulator before the milling.

19. A sensor for sequencing a long-chain molecule, the sensor comprising:
a fin field effect transistor, the source and drain being fins less than 1 nm thick and the gate having a microfluidic channel etched through it perpendicular to the fins.
